(19) United States
(12) Patent Application Publication
(10) Pub. No.: US 2023/0032632 A1
(43) Pub. Date: Feb. 2, 2023

(54) MEDICAL DEVICE HAVING A HOUSING WITH A DOOR THAT TRANSMITS OPTICAL SIGNALS

(71) Applicant: Fresenius Vial SAS, Brézins (FR)

(72) Inventors: Mathieu Paoli, La Murette (FR); Fabien Thomas, St Victor de Cessieu (FR); Jean-Marc Ulrich, St Jean de Moirans (FR); Rémy Wolff, Tullins (FR)

(21) Appl. No.: 17/758,119

(22) PCT Filed: Feb. 19, 2021
(86) PCT No.: PCT/EP2021/054095

§ 371 Date: Jun. 28, 2022

(30) Foreign Application Priority Data
Feb. 21, 2020 (EP) 20290022.1

(51) Int. Cl. A61M 5/50; A61M 5/142

(52) U.S. Cl. CPC A61M 5/5086; A61M 5/142; A61M 2205/587; A61M 2205/18

(57) ABSTRACT

Medical device having a housing with a door that transmits optical signals A medical device (1; 1′) comprises: a housing (10), a signal source (11) arranged at a section (100) of the housing (10), a control device (12) for controlling the emission of an optical signal by the signal source (11) in dependence of an operation of the medical device (1; 1′), and a door (13; 13) movably connected to the housing (10), wherein the section (100) of the housing (10) is covered by the door (13; 13) in a closed state of the door (12), and exposed in an open state of the door (13; 13′), the door (13; 13) comprising an optical device (14) adapted to transmit at least partially the optical signal through the door (13; 13′) when the door (13; 13) is in the closed state, such that the optical signal of the signal source (11) can be seen by an operator both when the door (13; 13′) is in the closed state and when the door (13; 13′) is the open state.

The invention relates to a medical device for providing a medical function, such as administering a medical fluid to a patient, and to a medical system with one or more of such medical devices.

Medical devices typically emit optical signals, in particular optical alarm signals, in dependence on the operation of the medical device. Specifically for medical devices, in many cases it needs to be assured that alarm signals can always be noticed by an operator of the medical device irrespective of an arrangement of movable parts of the medical device for safety reasons. The operator may be, e.g., a nurse or a physician.

US2019351138 refers to an infusion pump with tube loading guidance and configuration having light emitting diodes (LEDs) configured to indicate whether a tube is properly or improperly loaded at respective load points on the infusion pump when the door is open. Additional LEDs are positioned on the front door to indicate the pump is “ON” as well as flow direction.

On the other hand, a need has arisen to provide medical devices with a compact form factor. A compact form factor could allow to arrange a plurality of such medical devices within a given space. This would allow to use a larger number of medical devices at a certain place, as well as to provide a simplified access of the operator to each of the plurality of medical devices.

CN110604851 is related to a mounting equipment for storing infusion pumps. The mounting equipment comprises a control box having an alarm light located on front and sides of the control box. The alarm light includes an L-shaped light guide. The alarm can be seen from multiple angles.

US2010207768 further relates to a topical negative pressure system with status indication having LED and light guide. The light guide acts as a light pipe ducting light from the PCB mounted LED to the prominent position on the device case work.

It is an object of the instant invention to provide a medical device which allows for a compact design while providing a good visibility of optical signals.

This object is achieved by means of a medical device comprising the features of claim 1.

Accordingly, a medical device comprises a housing, a signal source, a control device, and a door. The signal source is arranged at a section of the housing. The control device is adapted for controlling the emission of an optical signal by the signal source in dependence of an operation of the medical device. The door is movably connected to the housing. The section of the housing is covered by the door when the door is in a closed state. The section of the housing is exposed when the door is in an open state. The door comprises an optical device adapted to transmit at least partially the optical signal of the signal source through the door when the door is in the closed state.

As a result, optical signals of the signal source can be seen by an operator both when the door is closed or in a closed state and when the door is opened or in an open state. The optical signal of the signal source is viewable, for instance by the operator, both when the door is in the closed state and when the door is in the open state. This allows to design the door such that it covers a large part of a side, e.g. the front panel, of the housing, or even the entire side, wherein signals from the signal source are always visible to an operator, irrespective of whether the door is closed or opened. For example, no additional area on the front panel besides the door needs to be provided for the signal source. Optionally, the door may (e.g., substantially) extend over the entire height and/or width of the housing. The optical device of the door allows the optical signals of the signal source at the housing to propagate from an inner side of the door (facing the housing) to an outer side of the door (facing away from the housing). Therefore, no additional light emitting element such as an LED is necessary to be provided at the door. An LED at the door would, e.g., be connected to the control device in the housing via a flexible PCB (flexible printed circuit board) which can have an enhanced risk of a failure. In addition, when using an LED on the door for emitting potentially safety-relevant signals, it could be necessary to make that part of the door water-tight, because medical devices usually need to be cleaned regularly.

Optionally, the control device is adapted to detect a predetermined state of operation of the medical device, and to control the emission of an optical alarm signal by the signal source in response to the detection of the predetermined state of operation. For example, the predetermined state of operation may be an error state, e.g. an occlusion of a fluid duct. Since the optical alarm signal may be seen by the operator irrespective of whether the door is opened or closed, the medical device allows to provide a high level of safety. The signal source may thus provide an alarm display. When the door is closed, the signal source together with the optical device form an alarm display. Alternatively or in addition, the predetermined state of operation may be an active state, or an inactive state; hence, a status display may be provided.

The medical device may be a pump, in particular an infusion pump for administering a medical fluid to a patient. An error state of an infusion pump may be specifically critical and necessary to be recognized by an operator quickly and consistently. As an example, the medical device may be a syringe pump. A syringe may be inserted into the syringe pump. Therefore, the housing can have a receptacle for receiving the syringe. Therein, the receptacle may at least partially be covered by the door when the door is in the closed state. The door may be opened to change the syringe. The syringe pump may be adapted to pump liquid out of the inserted syringe irrespective of whether the door is opened or closed. As such, the operator may open the door to check the syringe without interrupting the provision of the medical fluid while the optical signals of the signal source can be seen at any time.

The door may be designed such that, in the closed state, the door extends over the entire height (or substantially the entire height) of the housing. This may contribute to a compact design. The door may serve as front panel.

The signal source may comprise at least one translucent element, e.g. at least one light guide. Alternatively or in addition, the optical device comprises at least one translucent element, e.g. at least one light guide. For example, light may enter the respective light guide at a first side, and exit the light guide at a second side. The first and second sides may be opposite one another. The light guide allows a simple and robust design and, at the same time, an efficient use of the light of the optical signals.

Optionally at least one light guide is tapered. By this, light of the optical signals may be concentrated to be more clearly visible by the operator.

At least one light guide of the optical device may be aligned with at least one light guide of the signal source when the door is in the closed state. This allows only little losses of light between the two light guides. For example, the light guide of the optical device abuts at the light guide of the signal source when the door is closed. Both light guides may have a principle axis, wherein the principle axes are coaxial with one another when the door is closed.

At least one light guide of the signal source can be adapted to guide light emitted by at least one light emitting element (e.g., an LED) to at least one light guide of the optical device when the door is in the closed state. In other words, the light guide of the signal source may optically couple a light emitting element with a light guide of the optical device.

At least one light guide of the optical device may be adapted to guide light from the at least one light guide of the signal source to an exterior of the door when the door is in the closed state.

At least one side of at least one light guide of the signal source can be overmolded by material of the housing. This allows a simple manufacturing of the medical device as well as a robust design. Alternatively or in addition, at least one light guide of the signal source and the housing may be connected to one another in a fluid-tight manner. This allows a thorough cleaning of the medical device without water entering the housing at the signal source. The fluid-tight connection may particularly be provided by overmolding the at least one light guide of the signal source with material of the housing.

The at least one light guide may be made of PMMA, polycarbonate or MABS (methyl methacrylate acrylonitrile butadiene styrene), in particular MABS of the type TX-0520T. Optionally, all light guides of the signal source and the optical device may be made of one or more of those materials. These materials, particularly MABS of the type TX-0520T provide good light guiding properties and are suitable for surfaces of medical devices. The housing may be made of acrylonitrile butadiene styrene (ABS) what allows cohesion with the light guide material.

At least one surface of the at least one light guide can be polished, in particular mirror-polished. When the optical signals pass the one or more light guides, parts of the signals may get lost, e.g. due to absorption. Mirror-polishing surfaces (e.g., the lateral surfaces) of the light guide(s) allows to increase the transmitted portions of the signals.

In one embodiment the outer surfaces of the light guides are treated such, that light exiting the light guide is diffuse. For example, at least one of the surfaces is coated with a diffusing coating or is covered with a diffusing foil or the like. Outer surfaces mean the surfaces that are facing a user, standing in front of the infusion device, in particular with the door in closed state or an open state.

At least one light guide can have an outer surface that is rougher than another of its outer surfaces. The rougher surface may be matte. The surface with the increased roughness may be a surface visible to the operator. For example, an outer surface of the light guide may have an average roughness value Ra of between 4 and 10 micrometers, in particular of 4.5 micrometers or 9.0 micrometers.

According to an aspect, a medical system is provided. The medical system comprises a rack and at least one medical device according to any embodiment described herein being mounted or mountable on the rack.

The medical system may comprise a plurality of medical devices according to any embodiment described herein, wherein at least one of the medical devices is an infusion pump of the type of a syringe pump, and at least another one of the medical devices is an infusion pump of the type of a volumetric pump. Both types of infusion pumps each have a door of a similar size, while the housings may have different sizes. By the provision of the signal sources on the housings and the optical devices at the doors, it is possible align the positions of where signal light is emitted among the different medical devices. This allows the operator to take a quick look along a line over all of the plurality of medical devices in order to check for signals, in particular for alarm signals.

The idea underlying the invention shall subsequently be described in more detail with respect to the embodiments shown in the figures. Herein:

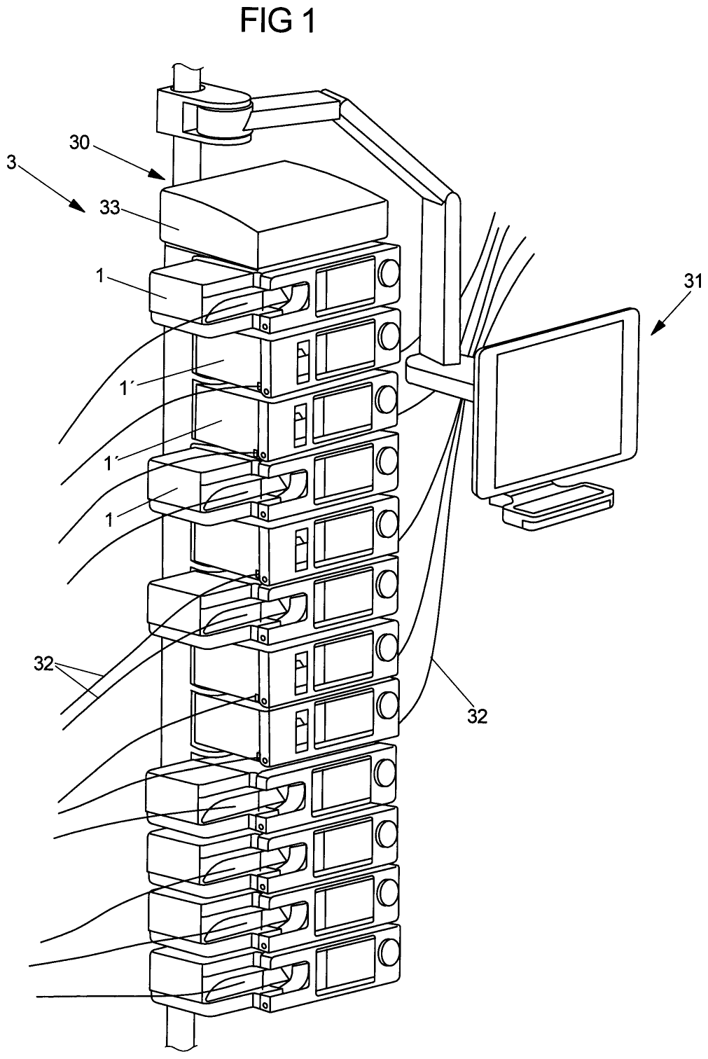

FIG. 1 shows a medical system with a plurality of medical devices;

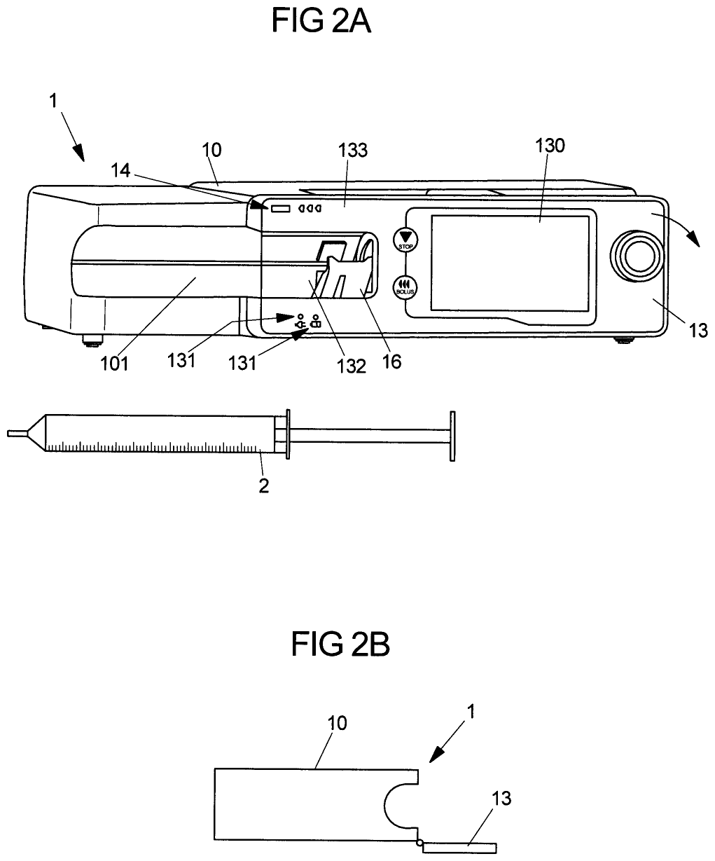

FIGS. 2A, 2B show views of an embodiment of a medical device being an infusion device in the shape of a syringe pump;

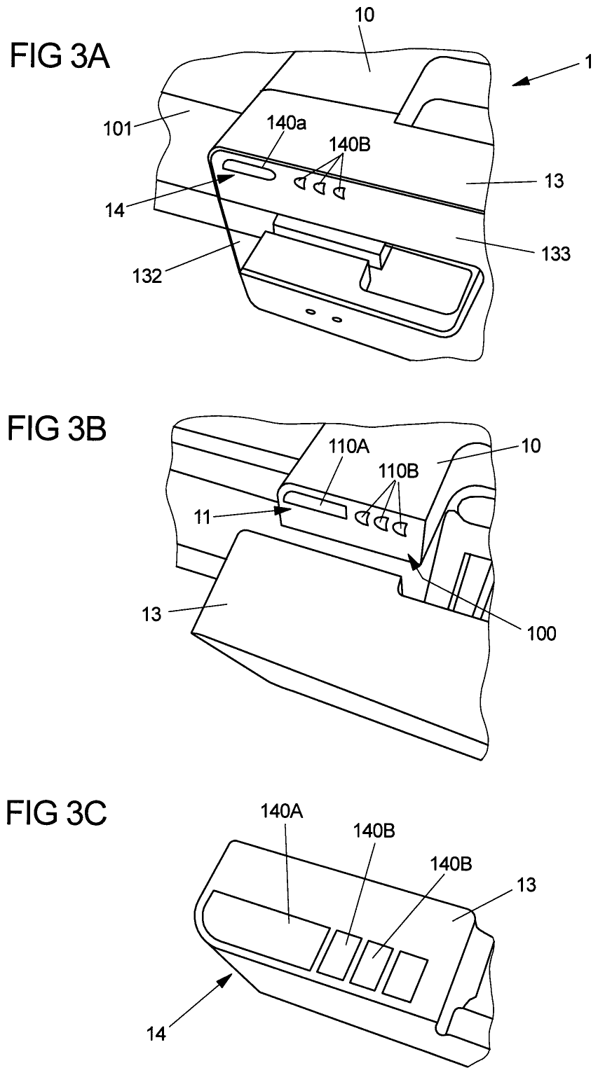

FIGS. 3A-3C show schematic views of a door of the infusion device according to FIG. 2A;

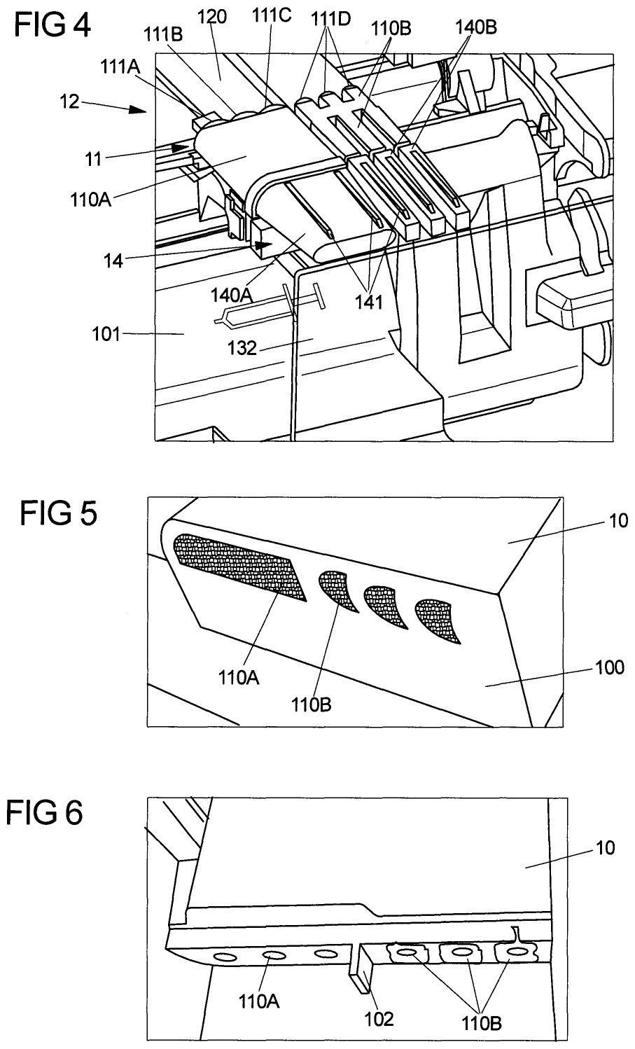

FIG. 4 shows a schematic view of several interior components of the infusion device according to FIG. 2A;

FIG. 5 shows a schematic view on outside faces of light guides of the infusion device according to FIG. 2A;

FIG. 6 shows a schematic view on inside faces of light guides of the infusion device according to FIG. 2A; and

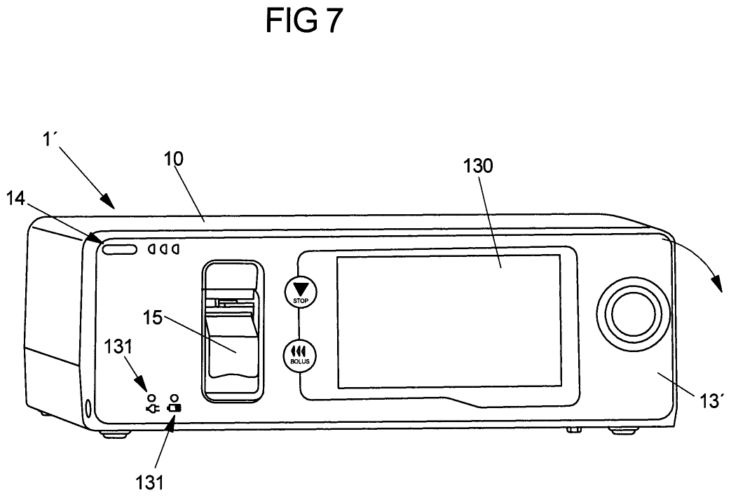

FIG. 7 shows a view of an embodiment of a medical device being an infusion device in the shape of a volumetric pump.

FIG. 1 shows a medical system 3 comprising a plurality of medical devices 1, 1′. The medical devices 1, 1′ are mounted on a rack 30 which itself, e.g., may have a stand or be fixed to a wall or the like. In the present case, each of the medical devices 1, 1′ is an infusion pump. According to FIG. 1, the medical system 3 comprises two different types of infusion pumps, namely medical devices 1 in the form of syringe pumps and medical devices 1′ in the form of volumetric pumps. The medical system 3 may be used, e.g., in a hospital.

The medical devices 1, 1′ are connected to an (optional) terminal 31 which could also be referred to as therapy manager. The terminal 31 allows an operator to control one or more of the medical devices 1, 1′ mounted on the rack 30. A communication interface 33 may be mounted on the rack 30 to enable a communication of the medical devices 1, 1′ among one another and/or with the terminal 31.

Each of the infusion pump medical devices 1, 1′ is adapted for administering a medical fluid to a patient. As such, depending on the type of medical fluid, it can be highly important to enable an operator, e.g. a nurse or a medical doctor, to check the status of operation of the medical devices 1, 1′. To this end, the medical devices 1, 1′ provide optical signals indicating, e.g., the operational status (e.g., whether the pump is active or inactive) and/or an alarm. One example for providing an alarm signal is an occlusion in a tube. This may impact or even impede the provision of the respective medical fluid. Such signals are important to be easily comprehensible and may be provided by illuminating a light of a certain color at a given location on the medical device 1, 1′.

Such signals should always be visible to the operator, irrespectively of the position of a movable part of the individual medical devices 1, 1′. In practice, one could provide an extension of a housing of a medical device that carries a signal source, and that protrudes such that it cannot be obstructed by a movable part of the medical device. On the other hand, however, there are many situations where a large number of medical devices are needed for one patient or in one place, e.g., in one treatment room. A compact form factor of the medical devices 1, 1′ directly allows to use a larger number of medical devices 1, 1′ within the same available volume, e.g. on the rack 30. The medical devices 1, 1′ shown in FIG. 1 have such a compact form factor and at the same time allow an operator to recognize signals irrespective of a movable part of the respective medical device 1, 1′, as will be described in more detail below. By this, it is possible to mount 10 or more, in the present example 12 medical devices 1, 1′ on the rack 30.

FIG. 2A shows an embodiment of a medical device 1 of the medical system 3, namely an infusion device in the shape of a syringe pump having a housing 10 and a receptacle 101 arranged on the housing 10 to receive a syringe 2 therein.

The syringe 2 comprises a cylindrical tube which, when installing the syringe 2 on the medical device 1, contains a medical liquid, for example a medication or a solution for the parenteral feeding, to be infused to a patient. The cylindrical tube is connected (e.g., via a connector) to an infusion line 32 (see FIG. 1) which may extend from the syringe 2 towards a patient for infusing the medical liquid to the patient.

The medical device 1 further comprises a door 13 that is movably connected to the housing, e.g., by means of hinges provided on a lower side of the door 13. The door 13 may thus be pivoted with respect to the housing 10 as indicated by an arrow in FIG. 1. FIG. 1 shows the door 13 in a closed state and the door may be pivoted to assume an open state (see FIG. 2B). In the closed state, the receptacle 101 is covered by the door 13, and in the opened state the receptacle 101 is exposed and accessible for an operator.

For installing the syringe 2 on the receptacle 101 of the medical device 1, first the door 13 needs to be opened. When the door 13 is in the open state, the cylindrical tube of the syringe 2 can be placed in the receptacle 101 and be mechanically connected to the housing 10 by means of a fixation device 16. By means of the fixation device 16, for example configured by a releasable clamp element, the cylindrical tube is secured within the receptacle 101 such that the cylindrical tube is held in position on the receptacle 101.

The syringe 2 comprises a piston which, for delivering medical fluid contained in the cylindrical tube, can be pushed into the cylindrical tube in a pushing direction. For this, the infusion device 1 comprises a pusher device movably arranged on the housing 10 and connected to a drive mechanism. For performing an infusion process the pusher device is then electrically actuated in the pushing direction to move the piston into the cylindrical tube for delivering the medical fluid contained in the cylindrical tube via the infusion line 32 (see FIG. 1) towards the patient. A window 132 is arranged in the door 13. This allows the operator to visually check the syringe 2 through the window.

As can be seen particularly in FIG. 2A, the door 13 substantially has the same height as the housing 10, i.e., in the closed state the door 13 covers a part of the housing 10 essentially over the entire height of the housing 10. By this, it is possible to arrange a large display 130 (in particular, a touch screen) on the door. Further, this design of the door 13 provides a clear impression that allows an operator to easily and quickly supervise a large number of medical devices 1, 1′. In the direction of a width of the housing 10 the door 13 covers more than half of the width of the housing 10, in the present example more than two thirds thereof.

It is worth noting that the syringe pump medical device 1 may both be operating while the door 13 is opened or closed. It is thus necessary that alarm and status signals can be seen irrespective of the state of the door 13.

The medical device 1 comprises an optical device 14 fixed to the door 13. The optical device 14 conveys light through the door 13. The optical device 14 comprises several translucent elements and guides light emitted by a signal source at the housing through the door 13 when the door is in the closed state as shown in FIG. 2A. The optical device 14 is arranged at an edge, presently at the upper left edge of the door 13. The optical device 14 will be described in more detail below.

The door 13 further comprises several status indicators 131, which, in the present case, serve to indicate whether or not the medical device 1 is provided with power (e.g., connected to a power socket or power supply), and an indicator for a charge level of a battery of the medical device 1. A light intensity of these indicators can be much lower than provided by the signal source and optical device 14. The door 13 serves as front panel of the medical device 1.

FIG. 3A is a perspective view of the part of the door 13 having the optical device 14. The optical device 14 comprises several translucent (in particular transparent) elements. In the example according to FIG. 3A, the optical device 14 comprises a plurality of light guides 140A, 140B. The light guides 140A, 140B are inserted into the door 13, so that in FIG. 3A only front faces of the light guides 140A, 140B are visible. The light guides 140A, 140B are adapted to let optical signals through the door 13. In other words, the light guides 140A, 140B transmit optical signals of the signal source 11, at least partially, through the door 13. It is worth noting that a part of the light of each signal may be lost in the passage through the light guides 140A, 140B. Optional measures to increase the efficiency of the transmission and to improve the clear visibility of the transmitted optical signals are described further below.

One of the light guides 140A serves to guide alarm signals and is larger than the other light guides 140B, the latter serving to guide status signals. The light guides 140A, 140B are not hollow. The light guides 140A, 140B of the optical device 14 each have a length that corresponds to the thickness of the door 13 at the place of installation of the light guides 140A, 140B.

FIG. 3B shows the door 13 in a partially opened state, and a section 100 of the housing 10 that is covered by the door 13 in the closed state (see FIG. 3A) is exposed and visible to an operator at an angle. As shown in FIG. 3B, a signal source 11 is arranged at the section 100 of the housing 10. The signal source 11 is adapted to emit optical signals. The signal source 11 comprises a plurality of light guides 110A, 110B having a similar (or the same) shapes and arrangement as the light guides 140A, 140B of the optical device 14.

When the door 13 is in an open state (e.g. in a fully opened state as shown in FIG. 2B) the section 100 of the housing 10 is exposed. When the section 100 of the housing 10 is exposed, display portions of the signal source 11 are directly visible to an operator. When the signal source 11 emits optical signals, the corresponding display portions are illuminated.

When the door 13 is in the closed state as shown in FIG. 2A, the optical signals from the signal source 11 are received by the optical device 14 on a side of the door 13 facing the housing 10, and emitted by the optical device 14 on a side of the door 13 facing away from the housing 10.

FIG. 3C shows the side of the door 13 that faces the housing 10 when the door 13 is in the closed position. Surfaces of the light guides 140A, 140B of the optical device 14 can be seen that face the housing 10 when the door 13 is in the closed position. In the closed position of the door 13 these surfaces of the light guides 140A, 140B are arranged adjacent exterior surfaces of the light guides 110A, 110B of the signal source 11.

FIG. 4 shows components of the medical device 1 wherein the housing 10 and parts of the door 13 are not shown. Specifically, FIG. 4 shows a control device 12 of the medical device 1. The control device 12 controls an operation of the medical device 1, e.g. it controls a motion of the pusher device. The control device 12 is adapted to determine a state of operation of the medical device 1, and it is adapted to determine whether the medical device 1 is in an error state. An error state may be an occlusion of a tube, e.g., the connected infusion line 32.

The control device 12 comprises a printed circuit board, PCB, 120. On the printed circuit board 120, a plurality of light-emitting elements 111A-111D of the signal source 11 are mounted and electrically connected. In the present example, each of the light-emitting elements is an LED. Three light-emitting elements 111A-111C are optically coupled to the light guide 110A for the alarm signal. These light-emitting elements 111A, 111B, 111C each are monochromatic LEDs adapted to emit blue, green and red light. Thus, an activation with selective intensity may provide different signal colors by additive color mixture. For example, when an alarm signal is to be emitted, a red light may be illuminated by only or predominantly activating the red LED. Furthermore, three light-emitting elements 111D are optically coupled with the light guides 110B for the status signal. All these light-emitting elements 111D are green LEDs. The control device 12 is adapted to activate these light-emitting elements 111D in an alternating manner (on/off) to indicate an ongoing pumping operation of the medical device 1. All (six) light-emitting elements 111A-111D are arranged along one straight line.

The light-emitting elements 111A-111D emit light predominantly along a major emission direction. The light guides 110A, 110B of the signal source 11 extend along this major emission direction. As can be seen in FIG. 4, when the door 13 is in the closed position, the light guides 140A, 140B of the optical device 14 at the door 13 are also arranged so as to extend along the major emission direction. Each one light guide 140A, 140B of the optical device 14 is aligned with a light guide 110A, 110B of the signal source 11. The light-emitting elements 111A-111D are designed so as to provide optical signals with an intensity that can be seen at a distance of 4 m in a 1600 lux ambient room.

The light guide 140A of the optical device 14 for the alarm signal (alternatively or in addition the other light guides 140B of the optical device 14) is tapered. Stated more precisely, its cross-sectional area decreases along the major emission direction. By this, light may be concentrated. By concentrating the light, a signal can be more clearly visible. For example, the shape of the light guide 140A may be trapezoidal.

The light guides 140A, 140B of the optical device 14 are press-fitted into through holes in the door 13. To ensure a tight connection, ribs 141 are provided on outer (lateral) surfaces of the light guides 140A, 140B.

The light guides 110A, 110B, 140A, 140B are made of PMMA, polycarbonate or MABS, in particular MABS of the type TX-0520T (Samsung).

Each of the signal source 11 and the optical device 14 comprises a (larger) light guide 110A, 140A for alarm signals, and three light guides 110B, 140B for the status signals.

FIG. 5 particularly shows that the light guides 110A, 110B of the signal source 11 which extend within the housing 10, have an exterior surface that is flush with an exterior surface of the housing 10 at said section 100. To ensure a fluid-tight enclosure of the internal electronic components, the light guides 110A, 11B of the signal source 11 are overmolded with material of the housing 11. The housing material may be, e.g., ABS. This material allows cohesion with the light guides 110A, 110B of the signal source 11 for waterproofness.

FIG. 5 further shows that the exterior surfaces of the light guides 110A, 110B of the signal source 11 are not even. Instead, they have an average roughness value Ra of between 4 and 10 micrometers. The roughness of the light guide 110A for the alarm signal may be different from the roughness of the light guide 110B for the status signal. For example, the light guide 110A for the alarm signal may have an average roughness Ra of 9.0 micrometers (charmille 39). The light guide 110B for the status signal may have an average roughness Ra of 4.5 micrometers (charmille 33). These values for the surface roughness have been determined to allow very wide viewing angles (e.g., spanning over 160 degrees or even 180 degrees) when the door 13 is open while at the same time allow an efficient coupling to the light guides 140A, 140B of the optical device 14 when the door 13 is closed.

The light guides 140A, 140B of the optical device 14 may have the roughness values on their exterior surfaces as described with reference to the light guides 110A, 110B of the signal source 11. However, in the example of FIG. 2A a foil 133 covers the front face of the door 13 and also covers the light guides 140A, 140B of the optical device 14. The foil is matte. By the provision of this foil 133 light of the optical signals is deflected. Therefore, the signals can also be seen at large viewing angles when the door 13 is closed.

The rough texture of light guides 110A, 110B may be made in an injection molding process with a corresponding tool, but could also be made by mechanical wear.

One or more, in particular all other surfaces of the light guides 110A, 110B, 140A, 140B that are not provided with a rough surface may be mirror-polished. Mirror-polishing one or more surfaces of the light guides 110A, 110B, 140A, 140B allow to minimize or even substantially avoid loss of light intensity. By this, a high efficiency of the signal transmission can be achieved.

FIG. 6 shows a view on the inner sides of the light guides 110A, 110B of the signal source 11 in a disassembled state. In an assembled state, the light-emitting elements 111A-111D are arranged adjacent these inner sides of the light guides 110A, 110B of the signal source 11. Between the light guide 110A for the alarm signals, and the light guides 110B for the status signals the housing 10 forms a rib 102. The rib 102 extends between the light-emitting elements 111A-111D (not shown in FIG. 6) to avoid light for a status signal to enter the light guide 110A for the alarm signals and vice-versa.

FIG. 7 shows another medical device 1′ of the medical system 3, namely an infusion device in the shape of a volumetric pump having a housing 10. Volumetric pumps are connected to a fluid supply and provide fluid to a patient via an infusion line 32 with an adjustable flow rate.

The medical device 1′ according to FIG. 7 has a door 13′ with a similar shape and size compared to the door 13 of the medical device 1 according to FIG. 2A (e.g., the same width). In particular, the medical device 1′ of FIG. 7 has a signal source 11, a control device 12 and an optical device 14 as (in particular identical to) the medical device 1 of FIG. 2A. Thus, reference is made to the explanations above.

The door 13′ extends over the entire front face of the housing 10 of the medical device 1′. A door lever 15 facilitates opening of the door 13′.

Referring again to FIG. 1, it now becomes even more apparent that the medical devices 1, 1′ are mounted on the rack 30 such that their optical devices 14 are arranged along a straight line. This homogeneity enables an operator of the medical system 3 to determine whether or not there is an alarm by just looking once along the straight line over the signal displays of all medical devices 1, 1′ (along the left edges of the doors 13, 13′).

The idea underlying the invention is not limited to the embodiments described above, but may be implemented in an entirely different fashion in entirely different embodiments.

An infusion device of the kind described herein may serve different purposes and may in particular be used to deliver a medical fluid such as a medication or a nutritional fluid, for example for the enteral or parenteral feeding, towards a patient.

### LIST OF REFERENCE NUMERALS

1; 1′ Medical device (e.g. infusion device)

10 Housing

100 Section

101 Receptacle

102 Rib

11 Signal source

110A, 110B Light guide

111A-111D Light emitting element

12 Control device

120 Printed circuit board

13; 13′ Door

130 Display

131 Status indicator

132 Window

133 Foil

14 Optical device

140A, 140B Light guide

141 Rib

15 Door lever

16 Fixation device

2 Syringe

3 Medical system

30 Rack

31 Terminal

32 Infusion line

33 Communication interface

1. A medical device, comprising:
a housing,
a signal source arranged at a section of the housing,
a control device for controlling an emission of an optical signal by the signal source in dependence of an operation of the medical device, and
a door movably connected to the housing, wherein the section of the housing is covered by the door in a closed state of the door, and exposed in an open state of the door, the door comprising an optical device adapted to transmit at least partially the optical signal through the door when the door is in the closed state, such that the optical signal of the signal source can be seen by an operator both when the door is in the closed state and when the door is the open state.

2. The medical device according to claim 1, wherein the control device is adapted to detect a predetermined state of operation of the medical device, and to control the emission of an optical alarm signal by the signal source in response to the detection of the predetermined state of operation.

3. The medical device according to claim 1 or 2, wherein the medical device is comprises an infusion pump for administering a medical fluid to a patient, wherein a receptacle for receiving a syringe or an infusion line is arranged at the housing, wherein the receptacle is at least partially covered by the door when the door is in the closed state.

4. The medical device according to claim 1, wherein in the closed state the door extends over the entire height of the housing.

5. The medical device according to claim 1, wherein the signal source and/or the optical device comprises at least one light guide.

6. The medical device according to claim 5, wherein the at least one light guide is tapered.

7. The medical device according to claim 5, wherein the signal source and the optical device each comprise at least one light guide, and the at least one light guide of the optical device is aligned with the at least one light guide of the signal source when the door is in the closed state.

8. The medical device according to one of claim 5, wherein the signal source and the optical device each comprise at least one light guide, and the at least one light guide of the signal source is adapted to guide light emitted by at least one light emitting element to the at least one light guide of the optical device when the door is in the closed state.

9. Medical The medical device according to claim 8, wherein the at least one light guide of the optical device is adapted to guide light from the at least one light guide of the signal source to an exterior of the door when the door is in the closed state.

10. The medical device according to claim 5, wherein at least one side of at least one light guide of the signal source is overmolded by material of the housing in a fluid-tight manner.

11. The medical device according to claim 5, wherein the at least one light guide is made of MABS, PMMA or polycarbonate.

12. The medical device according to claim 5, wherein at least one surface of at least one light guide is mirror-polished.

13. The medical device according to claim 5, wherein the at least one light guide has an outer surface with an average roughness value Ra of between 4 and 10 micrometers.

14. A medical system comprising a rack and at least one medical device according to claim 1 being mounted or mountable on the rack.

15. The medical system according claim 14, wherein the at least one of the medical devices comprises an infusion pump of the type of a syringe pump and is an infusion pump of the type of a volumetric pump.

16. The medical device according to claim 13, wherein the average roughness value Ra of the outer surface is 4.5 micrometers.
